(19) United States
(12) Patent Application Publication
(10) Pub. No.: US 2026/0102170 A1
(43) Pub. Date: Apr. 16, 2026

(54) COMPOSITE DEVICE FOR AUTOLOGOUS BONE HARVESTING AND BONE PROCESSING

(71) Applicant: THE FOURTH MEDICAL CENTER OF THE CHINESE PEOPLE'S LIBERATION ARMY GENERAL HOSPITAL, Beijing (CN)

(72) Inventors: Dong ZHANG, Beijing (CN); Taoguang WU, Beijing (CN); Yu JIANG, Beijing (CN); Qianjin YU, Beijing (CN); Chenxi YANG, Beijing (CN); Zina GU, Beijing (CN); Mengmeng HU, Beijing (CN); Xiaomeng YANG, Beijing (CN); Yong XIE, Beijing (CN); Jiantao LI, Beijing (CN); Zipeng ZHOU, Beijing (CN); Lihai ZHANG, Beijing (CN); Wei ZHANG, Beijing (CN)

(21) Appl. No.: 19/420,327

(22) Filed: Dec. 15, 2025

Related U.S. Application Data
(63) Continuation of application No. PCT/CN2025/119171, filed on Sep. 5, 2025.

(30) Foreign Application Priority Data
Jul. 12, 2024 (CN) 202410931271.2

(51) Int. Cl. A61B 17/16; A61B 17/17; A61B 90/00

(52) U.S. Cl. CPC A61B 17/1635; A61B 17/1606; A61B 17/17; A61B 2017/1602; A61B 2090/036

(57) ABSTRACT

The present invention relates to the technical field of bone processing, and in particular to a composite device for autologous bone harvesting and bone processing, including: a bone harvesting auxiliary device and a bone processing device. The bone harvesting auxiliary device includes: a bone harvesting caliper, a guide sleeve, and a depth-limiting Kirschner wire. The bone processing device includes: a bone box, a fastening assembly, and a cutting assembly. The composite device for autologous bone harvesting and bone processing in the present invention is capable of precise harvesting based on preoperative imaging measurements, thereby preventing resource waste. Intraoperatively, the device can obtain cancellous bone, unicortical bone, bicortical bone, and tricortical bone. In addition, various finished bone products of different shapes and sizes can be prepared according to requirements for a bone grafting area, such as bone granules, bone strips, bone chips, and bone blocks.

### CROSS-REFERENCE TO RELATED APPLICATIONS

This application is a continuation of International Application No. PCT/CN2025/119171, filed on Sep. 5, 2025, which claims priority to Chinese patent application No. 202410931271.2, titled “COMPOSITE DEVICE FOR AUTOLOGOUS BONE HARVESTING AND BONE PROCESSING”, filed on 12 Jul. 2024, the entire contents of which are incorporated herein by reference.

### FIELD OF TECHNOLOGY

The present invention relates to the technical field of bone processing, and in particular to a composite device for autologous bone harvesting and bone processing.

### BACKGROUND

Nonunion, bone defects, bone atrophy, osteonecrosis, impaired or difficult bone fusion (certain spinal instability, pseudarthrosis, or metabolic diseases), and conditions requiring orthopedic revision surgery are refractory diseases in orthopedic treatment, with an overall incidence rate exceeding 10%. Improper management may lead to long-term functional impairment or even disability in patients. The above orthopedic diseases usually require multiple large and complex surgeries, posing significant challenges to patients'quality of life and family finances.

Autologous bone harvesting and grafting is one of the most commonly used techniques in orthopedics, can simultaneously address issues of osteoinduction, osteogenesis, osteoconduction, and bone support, and is a “golden standard” for treating complex orthopedic conditions such as nonunion, bone defects, and orthopedic revision surgery.

In clinical practice, the autologous bone harvesting and grafting most commonly involves harvesting iliac bone. However, an amount of autologous bone available from the iliac crest is limited and often insufficient to meet the needs of grafting for large segmental bone defects. When a required amount exceeds 70 cc, it is often necessary to mix other bone graft materials. A composition ratio, contact surface, and physical form of a mixture of the iliac bone and other bone graft materials directly affect clinical efficacy of the autologous bone grafting.

In this case, bone strips, bone granules, and bone chips of suitable morphology can achieve optimal grafting results, thereby reducing an amount of autologous bone needed for harvesting. In addition, in the autologous bone harvesting, the most commonly used surgical instruments by clinicians are bone chisels and bone hammers, which often make it difficult to ensure precise bone harvesting and may easily cause iatrogenic fractures, bleeding, hematoma and infection, as well as injury to muscles and nerves. Further, in morphological processing of autologous bone, the most commonly used surgical instruments are bone scissors and bone chisels, which often make it difficult to ensure uniform shapes and sizes and are extremely time-consuming, and repeated cutting increases a risk of contamination. Therefore, there is an urgent clinical need for a simple and lightweight surgical instrument system for the autologous bone harvesting and bone processing.

### SUMMARY

In view of the above, the present invention proposes a composite device for autologous bone harvesting and bone processing, mainly to solve a problem of how to implement precise bone harvesting and process bone morphology as needed during autologous bone harvesting and processing, while reducing a difficulty in and risk of bone harvesting and processing.

In one aspect, the present invention provides a composite device for autologous bone harvesting and bone processing, including:
a bone harvesting auxiliary device and a bone processing device, where
the bone harvesting auxiliary device includes:
a bone harvesting caliper, including four caliper bars and four first knobs, each pair of the four caliper bars intersecting, and intersections being connected by the first knobs;
where the first knob is configured to adjust a fastening degree between two caliper bars and to set and plan a bone harvesting area;
guide sleeves, including four ones, with one end of each connected to the first knob for controlling a direction and gripping the bone harvesting caliper;
depth-limiting Kirschner wires, including four ones, disposed inside the guide sleeves for securing the bone harvesting caliper closely and firmly to the bone,
the bone processing device includes:
a bone box, with a partition plate disposed in the middle, and a bone drawer disposed at the bottom, and the bone drawer being configured to collect a finished bone product,
where a groove is provided on the partition plate;
a fastening assembly, including a compression rod and a push rod, where the compression rod is disposed on a side wall of the bone box and extends into the interior of the bone box, and the push rod is disposed on a top surface of the bone box and extends into the interior of the bone box, for securing the bone and preventing slippage and overturning;
a cutting assembly, including a rocker and a cutting blade, where one end of the rocker is disposed on a groove of the partition plate inside the bone box, and the rocker is located at an upper part of the bone drawer, the cutting blade is disposed on the rocker and is adjacent to an inner side wall of the bone box, and a length of the cutting blade is greater than a height of the bone box, so that the cutting blade has a lateral arcuate curvature; and
a thread is provided on an outer side wall of one end of the rocker inside the bone box, for assisting in pushing a bone block;
where the bone drawer is an L-shaped structural connecting plate, one end of the bone drawer is parallel to a bottom surface of the bone box, and the other end of the bone drawer is parallel to a side wall of the bone box.

In some embodiments of this application, a second through hole is provided in the first knob, one end of the second through hole protrudes outside the first knob, the second through hole is located at an outer side wall of one end outside the first knob, and a thread is provided on an inner side wall of the second through hole; and
the first knob is sequentially connected to one slider on each of the two slide rails, and the second through hole corresponds to positions of the first through holes in the two sliders.

In some embodiments of this application, the guide sleeve has a hollow structure, an outer side wall of one end of the guide sleeve is provided with an anti-slip knurling, an inner side wall of the other end of the guide sleeve is provided with a thread, the other end of the guide sleeve is threadedly connected to the first knob, and the first through hole in the first knob extends into the guide sleeve.

In some embodiments of this application, the depth-limiting Kirschner wire is a cylindrical bone pin, one end of the depth-limiting Kirschner wire is provided with a thread, a depth-limiting disc is further provided close to the end provided with the thread of the depth-limiting Kirschner wire, and a diameter of the depth-limiting disc is greater than a diameter of the second through hole; and
the depth-limiting Kirschner wire is disposed inside the guide sleeve, the end provided with the thread of the depth-limiting Kirschner wire faces the end provided with the thread of the guide sleeve, and the end provided with the thread of the depth-limiting Kirschner wire extends into the first knob.

In some embodiments of this application, both long edges of the caliper bar are provided with tapered beveled surfaces, and scales are provided on both the caliper bar and the first knobs.

In some embodiments of this application, the push rod includes:
a first connecting plate, disposed outside the bone box;
a second connecting plate, disposed on an outer side wall of the bone box;
a main spring, with both ends respectively connected to two opposite side walls of the first connecting plate and the second connecting plate;
a first connecting rod, with one end passing through the main spring and connected to one side wall of the first connecting plate, and the other end sequentially passing through the second connecting plate and the outer side wall of the bone box to extend into the interior of the bone box;
auxiliary springs, including several ones, with both ends of each respectively connected to two opposite side walls of the first connecting plate and the second connecting plate; and
a third connecting plate, disposed inside the bone box, with one side wall of the third connecting plate connected to the other end of the first connecting rod, and the other side wall of the third connecting plate provided with toothed protrusions.

In some embodiments of this application, two sets of compression rods are provided, including:
a fourth connecting plate, disposed at the top of the bone box;
a fifth connecting plate, disposed inside the bone box;
a sixth connecting plate, disposed inside the bone box, with an upper surface of the sixth connecting plate connected to a lower surface of the fifth connecting plate, and three side edges of the sixth connecting plate being in contact with corresponding inner side walls of the bone box;
a second connecting rod, with one end connected to a lower surface of the fourth connecting plate, the other end passing through a top surface of the bone box to extend into the interior of the bone box and connected to an upper surface of the fifth connecting plate, and a thread being provided on an outer side wall of the second connecting rod; and
a limiting connecting plate, which is an L-shaped structure and is disposed on one side of the sixth connecting plate.

In some embodiments of this application, the cutting assembly further includes:
a second knob, disposed on an outer side wall of the bone box close to the cutting blade, with scales provided on the second knob, and the second knob being configured to adjust a lateral arcuate curvature of the cutting blade.

In some embodiments of this application, the cutting assembly further includes a sleeve, firmly sleeved on the rocker, and the sleeve passes through a side wall of the bone box away from the push rod; and
the other end of the rocker sequentially passes through the sleeve, an outer side wall of the bone box, and the second knob, and a grip handle is provided at the other end of the rocker.

In some embodiments of this application, the cutting blade is a flat arcuate blade or a curved tooth blade.

Compared with the prior art, the present invention has the following beneficial effects.

The bone harvesting auxiliary device in the present invention is characterized in that the bone harvesting caliper can be freely assembled into various shapes, enabling precise extraction of bone blocks of suitable shapes and volume according to preoperative imaging data and intraoperative requirements. The depth-limiting Kirschner wire has a compression function, allowing the bone harvesting caliper to closely adhere to a bone surface, thereby preventing iatrogenic fractures caused by uneven use of bone chisels or excessive force, and reducing a risk of bone debris splashing. The caliper bar is provided with a tapered surface, which is similar to a periosteal elevator, and can flexibly push a periosteum, reduce bleeding, and improve the efficiency of exposure in a surgical area. In addition, the guide sleeve or the depth-limiting Kirschner wire is connected to shield important anatomical structures, protecting blood vessels, nerves, and muscles, reducing a risk of iatrogenic injury, replacing retractors, saving manpower, and achieving smaller incisions and minimally invasive goals. Even in cases where a patient has thick adipose tissue or deep wounds, single-person operation is possible without an additional retractor, reducing a need for surgical assistants.

Further, the bone processing device in the present invention can prepare various types and sizes of finished bone products, such as bone blocks, bone chips, bone strips, and bone granules, according to a morphology of a grafting area.

In addition, the composite device for autologous bone harvesting and bone processing in the present invention can perform precise material acquisition based on preoperative imaging measurements, avoiding resource waste. Intraoperatively, the device can obtain cancellous bone, unicortical bone, bicortical bone, and tricortical bone. In addition, various finished bone products of different shapes and sizes can be prepared according to requirements for a bone grafting area.

### BRIEF DESCRIPTION OF THE DRAWINGS

With reference to detailed descriptions in preferred implementations in the following descriptions, various other advantages and benefits become clear to a person of ordinary skills in the art. The accompanying drawings are merely used to show preferred implementations, and are not considered as limitations to the present invention. In the drawings:

FIG. 1 is a schematic structural diagram of a bone harvesting caliper according to an embodiment of the present invention;

FIG. 2 is a schematic structural diagram of a guide sleeve and a depth-limiting Kirschner wire according to an embodiment of the present invention;

FIG. 3 is a sectional view of a bone processing device according to an embodiment of the present invention;

FIG. 4 is a front view of the bone processing device according to an embodiment of the present invention; and

FIG. 5 is a schematic structural diagram of a cutting blade according to an embodiment of the present invention.

### DESCRIPTION OF THE EMBODIMENTS

Exemplary embodiments of the present disclosure will be described below in further detail with reference to the accompanying drawings. Although the accompanying drawings show exemplary embodiments of the present disclosure, it should be understood that the present disclosure can be implemented in various forms and shall not be limited by embodiments described herein. On the contrary, these embodiments are provided to enable a more thorough understanding of the present disclosure and to fully convey the scope of the present disclosure to those skilled in the art. It should be noted that embodiments in the present invention and the features in the embodiments may be mutually combined in the case of no conflict. The present invention is described in detail in the following with reference to the accompanying drawings by using embodiments.

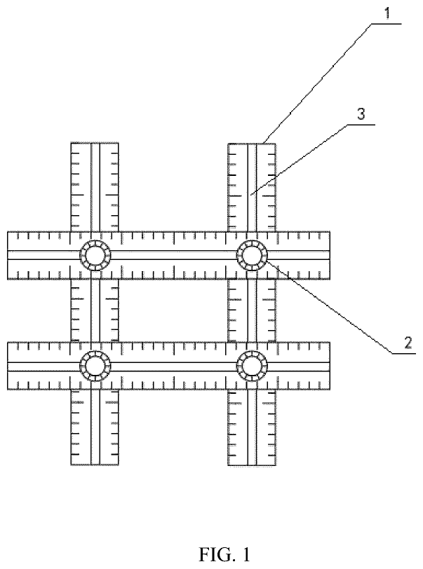

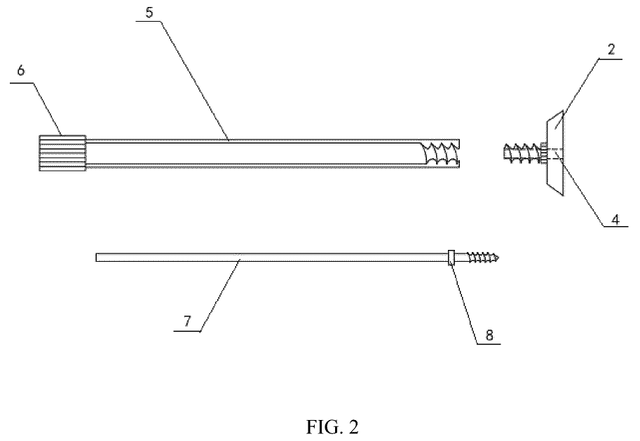

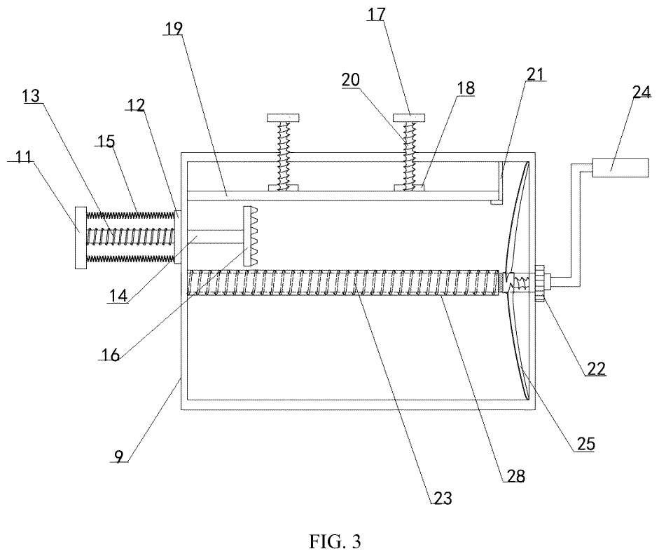

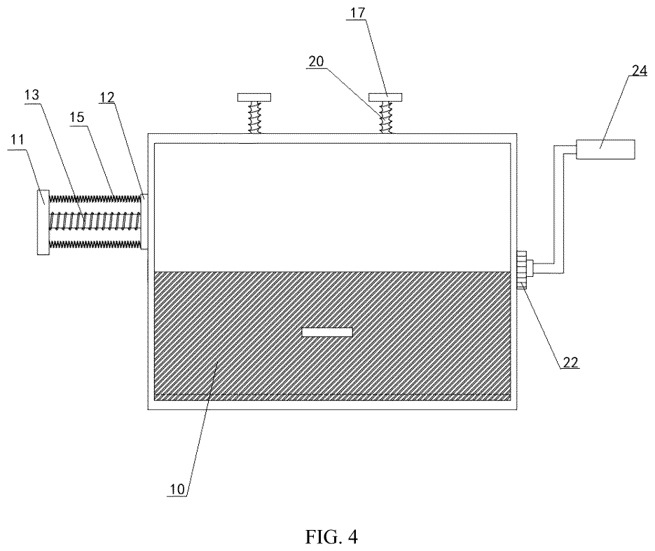

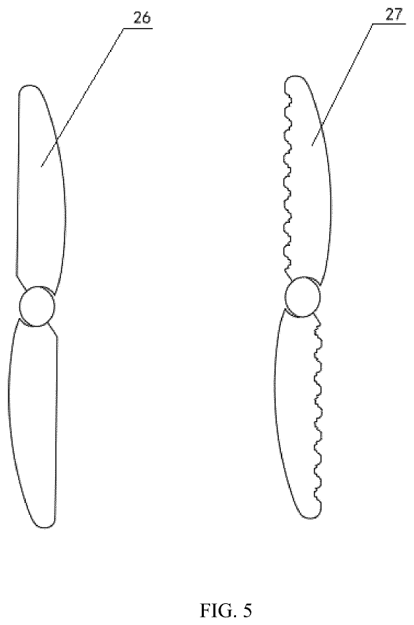

Refer to FIG. 1 to FIG. 5. This embodiment provides a composite device for autologous bone harvesting and bone processing, including:
a bone harvesting auxiliary device and a bone processing device.

The bone harvesting auxiliary device includes:
a bone harvesting caliper, including four caliper bars 1 and four first knobs 2, each pair of the four caliper bars 1 intersecting, and intersections being connected by the first knobs 2,
where the first knob 2 is configured to adjust a fastening degree between two caliper bars 1 and to set and plan a bone harvesting area;
guide sleeves 5, including four ones, with one end of each connected to the first knob 2 for controlling a direction and gripping the bone harvesting caliper; and
depth-limiting Kirschner wires 7, including four ones, disposed inside the guide sleeves 5 for fastening the bone harvesting caliper closely and firmly to the bone.

The bone processing device includes:
a bone box 9, with a partition plate 28 disposed in the middle, and a bone drawer 10 disposed at the bottom, and the bone drawer 10 being configured to collect a finished bone product,
where a groove is provided on the partition plate 28;
a fastening assembly, including a compression rod and a push rod, where the compression rod is disposed on a side wall of the bone box 9 and extends into the interior of the bone box 9, and the push rod is disposed on a top surface of the bone box 9 and extends into the interior of the bone box 9;
a cutting assembly, including a rocker 23 and a cutting blade 25, where one end of the rocker 23 is disposed on a groove of the partition plate 28 inside the bone box 9, and the rocker 23 is located at an upper part of the bone drawer 10, the cutting blade 25 is disposed on the rocker 23 and is adjacent to an inner side wall of the bone box 9, and a length of the cutting blade 25 is greater than a height of the bone box 9, so that the cutting blade 25 has a lateral arcuate curvature; and
a thread is provided on an outer side wall of one end of the rocker 23 inside the bone box 9, for assisting in pushing a bone block.

The bone drawer 10 is an L-shaped structural connecting plate, one end of the bone drawer 10 is parallel to a bottom surface of the bone box 9, and the other end of the bone drawer 10 is parallel to a side wall of the bone box 9.

It can be understood that the composite device for autologous bone harvesting and bone processing provided in this embodiment combines the bone harvesting auxiliary device and the bone processing device, implementing easy autologous bone harvesting and efficient processing.

The design of the bone harvesting caliper enables a doctor to accurately measure and locate a bone block to be extracted. The first knob 2 is adjusted, so that the doctor can easily control a degree of fastening between the caliper bars 1 to accommodate bone blocks of different shapes and sizes. The guide sleeve 5 and the depth-limiting Kirschner wire 7 ensure precision and safety during bone harvesting, preventing accidental injury to blood vessels, nerves, muscles, and iatrogenic fractures during operation.

The bone box 9 is designed to provide enclosed space for storing and processing the bone block, and the bone drawer 10 is disposed to enable the doctor to easily collect and process the finished bone product. The compression rod and the push rod of the fastening assembly can effectively fasten and move the bone block, making a subsequent cutting operation more precise and smooth. The rocker 23 and the cutting blade 25 in the cutting assembly are designed to allow the doctor to easily adjust a cutting thickness and angle, thereby implementing efficient cutting of the bone block.

The entire device ingeniously integrates two steps of bone harvesting and bone processing, significantly enhancing the safety and precision of surgery through accurate bone harvesting assistance and efficient bone processing.

In a specific embodiment of this application, the bone harvesting caliper further includes:
slide rails 3, each disposed in the middle of each of the caliper bars 1 and parallel to a long edge of each of the caliper bars 1, and having hollow structures; and
sliders, including eight ones, two of the sliders being slidably disposed on one of the slide rails 3, and a first through hole being provided in the slider, with a thread provided on an inner side wall of the first through hole.

In a specific embodiment of this application, a second through hole 4 is provided in the first knob 2, and one end of the second through hole 4 protrudes outside the first knob 2. The second through hole 4 is located at an outer side wall of one end outside the first knob 2, and a thread is provided on an inner side wall of the second through hole 4.

The first knob 2 is sequentially connected to one slider on each of the two slide rails 3, and the second through hole 4 corresponds to positions of the first through holes in the two sliders.

It can be understood that, for the bone harvesting caliper, more precise and flexible planning of an area and bone harvesting operation are implemented through the design of addition of the slide rails 3 and sliders, and ingenious combination of the first knobs 2 with these components.

First, the provision of the slide rails 3 allows the sliders to slide on the caliper bars 1, enabling the doctor to adjust positions of the sliders as needed during bone harvesting, thereby controlling the use of the caliper bars 1 more precisely. The slide rails 3 are parallel to long edges of the caliper bars 1, ensuring stability of slider movement and more accurate measurements.

Second, the first through hole provided in the slider and the thread on the inner side wall are designed to be possibly connected to the first knob 2. This design enables the connection between the slider and the first knob 2 to be more secure and less likely to detach, thereby improving the safety of use of the caliper bars 1.

Further, the second through hole 4 in the first knob 2 not only facilitates a connection of the first knob 2 with other components and prevents loosening, but also enables the caliper bars 1 to be more stable during use, improving operational accuracy.

Finally, the first knob 2 is sequentially connected to one slider on each of the two slide rails 3, enabling the caliper bars 1 to be used more flexibly. What's more, the second through hole 4 corresponds to positions of the first through holes in the two sliders, ensuring the accuracy and stability of the connection.

In a specific embodiment of this application, the guide sleeve 5 has a hollow structure, and an outer side wall of one end of the guide sleeve 5 is provided with an anti-slip knurling 6. An inner side wall of the other end of the guide sleeve 5 is provided with a thread. The other end of the guide sleeve 5 is threadedly connected to the first knob 2, and the first through hole in the first knob 2 extends into the guide sleeve 5.

In a specific embodiment of this application, the depth-limiting Kirschner wire 7 is a cylindrical bone pin, and a thread is provided at one end of the depth-limiting Kirschner wire 7. A depth-limiting disc 8 is further provided close to the end provided with the thread of the depth-limiting Kirschner wire 7, and a diameter of the depth-limiting disc 8 is greater than a diameter of the second through hole 4.

The depth-limiting Kirschner wire 7 is disposed inside the guide sleeve 5, the end provided with the thread of the depth-limiting Kirschner wire 7 faces the end provided with the thread of the guide sleeve 5, and the end provided with the thread of the depth-limiting Kirschner wire 7 extends into the first knob 2.

In a specific embodiment of this application, both long edges of the caliper bar 1 are provided with tapered beveled surfaces, and scales are provided on both the caliper bars 1 and the first knobs 2.

It can be understood that the guide sleeve 5 in this embodiment is a hollow structure, with the anti-slip knurling 6 provided on the outer side wall at one end, which can increase stability during operation. An inner side wall on the other end is threaded and can be screwed into the first knob 2, to match the thread on the outer side wall of the first knob 2 for connection with the first knob 2. The other end of the guide sleeve 5 is threadedly connected to the first knob 2, allowing a length of the entire device to be adjusted during use. The depth-limiting Kirschner wire 7 is a cylindrical bone pin, with a thread provided at one end and the depth-limiting disc 8 provided close to a threaded portion at the other end. A diameter of the depth-limiting disc 8 is greater than a diameter of the second through hole 4, which can prevent the depth-limiting Kirschner wire 7 from penetrating too deeply into the bone during use. The depth-limiting Kirschner wire 7 is disposed inside the guide sleeve 5, and the end provided with the thread faces the end provided with the thread of the guide sleeve 5, so that the depth-limiting Kirschner wire 7 can move within the guide sleeve 5 by rotating the first knob 2.

Both long edges of the caliper bar 1 have tapered beveled surfaces, facilitating operation during surgery. Scales are provided on both the caliper bars 1 and the first knobs 2, enabling precise measurement and positioning during surgery.

In a specific embodiment of this application, the push rod includes:
a first connecting plate 11, disposed outside the bone box 9;
a second connecting plate 12, disposed on an outer side wall of the bone box 9;
a main spring 13, with both ends respectively connected to two opposite side walls of the first connecting plate 11 and the second connecting plate 12;
a first connecting rod 14, with one end passing through the main spring 13 and connected to one side wall of the first connecting plate 11, and the other end sequentially passing through the second connecting plate 12 and the outer side wall of the bone box 9 to extend into the interior of the bone box 9;
auxiliary springs 15, including several ones, with both ends of each respectively connected to two opposite side walls of the first connecting plate 11 and the second connecting plate 12; and
a third connecting plate 16, disposed inside the bone box 9, with one side wall of the third connecting plate 16 connected to the other end of the first connecting rod 14, and the other side wall of the third connecting plate 16 provided with toothed protrusions.

It can be understood that, a stable connection is established between the push rod and the bone box 9 through the design of the first connecting plate 11 and the second connecting plate 12 in this embodiment. This structure not only ensures the stability of the push rod on the bone box 9, but also allows the push rod to maintain a stable motion trajectory when subjected to an external force. The main spring 13 and the auxiliary springs 15 are disposed to provide the push rod with elasticity, enabling the push rod to freely expand and contract within a specific range. This design not only enables the push rod to be operated more flexibly, but also can effectively absorb and buffer an external impact force, protecting both the bone box 9 and the push rod from being damaged. The first connecting rod 14 passes through the main spring 13 and the second connecting plate 12, and then extends into the bone box 9 to be connected to the third connecting plate 16. This design allows movement of the push rod to be smoothly transmitted to the interior of the bone box 9, ensuring continuity and stability of operation. The toothed protrusions on the third connecting plate 16 can be customized according to specific requirements, and the toothed protrusions can be configured to cooperate with other structures inside the bone box 9.

In a specific embodiment of this application, two sets of compression rods are provided, including:
a fourth connecting plate 17, disposed at the top of the bone box 9;
a fifth connecting plate 18, disposed inside the bone box 9;
a sixth connecting plate 19, disposed inside the bone box 9, with an upper surface of the sixth connecting plate 19 connected to a lower surface of the fifth connecting plate 18, and three side edges of the sixth connecting plate 19 being in contact with corresponding inner side walls of the bone box 9;
a second connecting rod 20, with one end connected to a lower surface of the fourth connecting plate 17, the other end passing through a top surface of the bone box 9 to extend into the interior of the bone box 9 and connected to an upper surface of the fifth connecting plate 18, and a thread being provided on an outer side wall of the second connecting rod 20; and
a limiting connecting plate 21, which is an L-shaped structure and is disposed on one side of the sixth connecting plate 19.

It can be understood that, through the combination of the fourth connecting plate 17, the fifth connecting plate 18, and the sixth connecting plate 19 in this embodiment, a robust supporting structure is formed. The sixth connecting plate 19 is designed particularly ingeniously, which is closely connected to the fifth connecting plate 18, and three side edges of which also contact inner side walls of the bone box 9, greatly enhancing connection stability between the compression rod and the bone box 9 and the overall rigidity of the structure. The second connecting rod 20 is designed to allow the doctor to make adjustments. Because a thread is provided on an outer side wall of the second connecting rod 20, this means that the thread can be matched with a corresponding threaded interface to implement a precise lifting or fastening function. This design enables a height or position of the compression rod to be adjusted according to actual needs, thereby adapting to different usage scenarios or operational requirements. The limiting connecting plate 21 is designed, to further enhance the functionality of the compression rod. As an L-shaped structure, the limiting connecting plate 21 can effectively prevent a material inside the bone box 9 from overflowing or shifting during compression. In addition, the limiting connecting plate 21 can also cooperate with other components to ensure that the material inside the bone box 9 is subjected to a uniform compressive force, thereby improving production efficiency and product quality.

In a specific embodiment of this application, the cutting assembly further includes:
a sleeve, firmly sleeved on the rocker 23, and the sleeve passing through a side wall of the bone box 9 away from the push rod;
a second knob 22, disposed at one end of the sleeve passing through the bone box 9, with scales provided on the second knob 22, where the second knob 22 is configured to adjust a lateral arcuate curvature of the cutting blade 25; and
the other end of the rocker 23 sequentially passes through the sleeve, an outer side wall of the bone box 9, and the second knob 22, and a grip handle 24 is provided at the other end of the rocker 23.

In a specific embodiment of this application, the cutting blade 25 is a flat arcuate blade 26 or a curved tooth blade 27.

It can be understood that the cutting assembly in this embodiment is equipped with the second knobs 22. The second knob 22 is located on the outer side wall of the bone box 9 and close to a position of the cutting blade 25. The scales are provided on the second knob 22, and the doctor can adjust the lateral arcuate curvature of the cutting blade 25 by rotating the knobs. This design enables the cutting blade 25 to be more flexible and allows adjustment according to different cutting requirements and material characteristics, thereby improving cutting precision and efficiency. The other end of the rocker 23 passes through the outer side wall of the bone box 9 and the second knob 22, and the grip handle 24 is provided at the end of the rocker, providing more easy operation for the doctor. The doctor can easily control the movement of the rocker 23 by holding the grip handle 24, thereby controlling a cutting thickness and angle of the cutting blade 25. The design of the grip handle 24 also increases operational comfort and stability.

Specifically, a tightening degree of the second knob 22 is adjusted to adjust a distance between one end of the sleeve where the second knob 22 is located and the other end of the sleeve, thereby adjusting the lateral arcuate curvature of the cutting blade 25.

Specifically, the cutting blade 25 may be a flat arcuate blade 26 or a curved tooth blade 27. The diversity enables the cutting assembly to accommodate different cutting requirements. The doctor can select a proper type of cutting blade 25 according to an actual situation to improve the cutting effect.

Specifically, a workflow of the bone harvesting auxiliary device in this embodiment is as follows: first, exposing a bone harvesting area, connecting a guide sleeve 5 to a bone harvesting caliper, and using a tapered beveled surface on a caliper bar 1, which resembles a “periosteal elevator,” to bluntly push away muscle and periosteum, and preliminarily shielding surrounding tissues by using the guide sleeve 5; subsequently, according to a preoperative plan and intraoperative requirements, adjusting a knob of the bone harvesting caliper, selecting a proper caliper shape based on a scale and angle of the caliper, simulating the bone harvesting area, and performing pre-tightening; and next, designing and determining the shape of the bone harvesting caliper, screwing in a depth-limiting Kirschner wire 7, and using a thin bone knife to harvest bone in a central bone harvesting area or sliding area.

Specifically, a workflow of the bone processing device in this embodiment is as follows: placing a bone block into a bone box 9, so that the bone block is located on the rocker 23, and fastening the bone block using two sets of compression rods and push rods from above to prevent the bone block from overturning and sliding; selecting a proper cutting blade 25 and adjusting a second knob 22 to select a proper scale, namely, a thickness of a finished bone product; and then, swinging a grip handle 24 on the rocker 23 to perform cutting, and collecting cuttings from an area of a bone drawer 10. If bone granules are needed, a bone strip is repeatedly placed into the bone box 9 for multiple cuttings.

Finally, it should be noted that the above embodiments are only intended to illustrate and not to limit the technical solutions of the present invention. Although the present invention has been described in detail with reference to the above embodiments, those skilled in the art should understand that modifications or equivalent replacements may still be made to the specific implementations of the present invention without departing from the spirit and scope of the invention. Any modifications or equivalent replacements that do not depart from the spirit and scope of the present invention shall be included within the scope of protection of the claims of the present invention.

What is claimed is:

1. A composite device for autologous bone harvesting and bone processing, comprising:
a bone harvesting auxiliary device and a bone processing device, wherein
the bone harvesting auxiliary device comprises:
a bone harvesting caliper, comprising four caliper bars and four first knobs, each pair of the four caliper bars intersecting, and intersections being connected by the first knobs;
wherein the first knob is configured to adjust a fastening degree between two caliper bars and to set and plan a bone harvesting area and size;
a guide sleeve, comprising four ones, one end of each being connected to the first knob; and
depth-limiting Kirschner wires, comprising four ones, and disposed inside the guide sleeve;
the bone processing device comprises:
a bone box, with a partition plate disposed in the middle, and a bone drawer disposed at the bottom, and the bone drawer being configured to collect a finished bone product,
wherein a groove is provided on the partition plate;
a fastening assembly, comprising a compression rod and a push rod, wherein the compression rod is disposed on a side wall of the bone box and extends into the interior of the bone box, and the push rod is disposed on a top surface of the bone box and extends into the interior of the bone box;
a cutting assembly, comprising a rocker and a cutting blade, wherein one end of the rocker is disposed on a groove of the partition plate inside the bone box, and the rocker is located at an upper part of the bone drawer, the cutting blade is disposed on the rocker and is adjacent to an inner side wall of the bone box, and a length of the cutting blade is greater than a height of the bone box, so that the cutting blade has a lateral arcuate curvature; and
a thread is provided on an outer side wall of one end of the rocker inside the bone box,
wherein the bone drawer is an L-shaped structural connecting plate, one end of the bone drawer is parallel to a bottom surface of the bone box, and the other end of the bone drawer is parallel to a side wall of the bone box.

2. The composite device for autologous bone harvesting and bone processing according to claim 1, wherein the bone harvesting caliper further comprises:
slide rails, each disposed in the middle of each of the caliper bars and parallel to a long edge of each of the caliper bars, and having hollow structures; and
sliders, comprising eight ones, two of the sliders being slidably disposed on one of the slide rails, and a first through hole being provided in the slider, with a thread provided on an inner side wall of the first through hole.

3. The composite device for autologous bone harvesting and bone processing according to claim 2, wherein a second through hole is provided in the first knob, one end of the second through hole protrudes outside the first knob, the second through hole is located at an outer side wall of one end outside the first knob, and a thread is provided on an inner side wall of the second through hole; and
the first knob is sequentially connected to one slider on each of the two slide rails, and the second through hole corresponds to positions of the first through holes in the two sliders.

4. The composite device for autologous bone harvesting and bone processing according to claim 3, wherein the guide sleeve has a hollow structure, an outer side wall of one end of the guide sleeve is provided with an anti-slip knurling, an inner side wall of the other end of the guide sleeve is provided with a thread, the other end of the guide sleeve is threadedly connected to the first knob, and the first through hole in the first knob extends into the guide sleeve.

5. The composite device for autologous bone harvesting and bone processing according to claim 4, wherein the depth-limiting Kirschner wire is a cylindrical bone pin, one end of the depth-limiting Kirschner wire is provided with a thread, a depth-limiting disc is further provided close to the end provided with the thread of the depth-limiting Kirschner wire, and a diameter of the depth-limiting disc is greater than a diameter of the second through hole; and
the depth-limiting Kirschner wire is disposed inside the guide sleeve, the end provided with the thread of the depth-limiting Kirschner wire faces the end provided with the thread of the guide sleeve, and the end provided with the thread of the depth-limiting Kirschner wire extends into the first knob.

6. The composite device for autologous bone harvesting and bone processing according to claim 1, wherein both long edges of the caliper bar are provided with tapered beveled surfaces, and scales are provided on both the caliper bar and the first knobs.

7. The composite device for autologous bone harvesting and bone processing according to claim 1, wherein the push rod comprises:
a first connecting plate, disposed outside the bone box;
a second connecting plate, disposed on an outer side wall of the bone box;
a main spring, with both ends respectively connected to two opposite side walls of the first connecting plate and the second connecting plate;
a first connecting rod, with one end passing through the main spring and connected to one side wall of the first connecting plate, and the other end sequentially passing through the second connecting plate and the outer side wall of the bone box to extend into the interior of the bone box;
auxiliary springs, comprising several ones, with both ends of each respectively connected to two opposite side walls of the first connecting plate and the second connecting plate; and
a third connecting plate, disposed inside the bone box, with one side wall of the third connecting plate connected to the other end of the first connecting rod, and the other side wall of the third connecting plate provided with toothed protrusions.

8. The composite device for autologous bone harvesting and bone processing according to claim 1, wherein two sets of compression rods are provided, comprising:
a fourth connecting plate, disposed at the top of the bone box;
a fifth connecting plate, disposed inside the bone box;
a sixth connecting plate, disposed inside the bone box, with an upper surface of the sixth connecting plate connected to a lower surface of the fifth connecting plate, and three side edges of the sixth connecting plate being in contact with corresponding inner side walls of the bone box;
a second connecting rod, with one end connected to a lower surface of the fourth connecting plate, the other end passing through a top surface of the bone box to extend into the interior of the bone box and connected to an upper surface of the fifth connecting plate, and a thread being provided on an outer side wall of the second connecting rod; and
a limiting connecting plate, which is an L-shaped structure and is disposed on one side of the sixth connecting plate.

9. The composite device for autologous bone harvesting and bone processing according to claim 1, wherein the cutting assembly further comprises:
a sleeve, firmly sleeved on the rocker, and the sleeve passing through a side wall of the bone box away from the push rod;
a second knob, disposed at one end of the sleeve passing through the bone box, with scales provided on the second knob, wherein the second knob is configured to adjust a lateral arcuate curvature of the cutting blade; and
the other end of the rocker sequentially passes through the sleeve, an outer side wall of the bone box, and the second knob, and a grip handle is provided at the other end of the rocker.

10. The composite device for autologous bone harvesting and bone processing according to claim 1, wherein the cutting blade is a flat arcuate blade or a curved tooth blade.
